(19) United States
(12) Patent Application Publication
(10) Pub. No.: US 2007/0281073 A1
(43) Pub. Date: Dec. 6, 2007

(54) Enhanced adhesion of drug delivery coatings on stents

(76) Inventors: David C. Gale, San Jose, CA (US); Daniel Castro, Santa Clara, CA (US); Timothy A. Limon, Cupertino, CA (US); Bin Huang, Pleasanton, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/446,489

(22) Filed: Jun. 1, 2006

(51) Int. Cl. A61L 33/00

(52) U.S. Cl. 427/2.25

(57) ABSTRACT

Methods of enhancing adhesion of drug delivery coatings on stents are disclosed.

### BACKGROUND OF THE INVENTION

1. Field of the Invention

This invention relates to drug delivery stents and methods for coating stents.

2. Description of the State of the Art

This invention relates to radially expandable endoprostheses, that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices, that function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of scaffolding that includes a pattern or network of interconnecting structural elements or struts, formed from wires, tubes, or sheets of material rolled into a cylindrical shape. This scaffolding gets its name because it physically holds open and, if desired, expands the wall of the passageway. Typically, stents are capable of being compressed or crimped onto a catheter so that they can be delivered to and deployed at a treatment site. Delivery includes inserting the stent through small lumens using a catheter and transporting it to the treatment site. Deployment includes expanding the stent to a larger diameter once it is at the desired location. Mechanical intervention with stents has reduced the rate of restenosis as compared to balloon angioplasty. Yet, restenosis remains a significant problem. When restenosis does occur in the stented segment, its treatment can be challenging, as clinical options are more limited than for those lesions that were treated solely with a balloon.

Stents are used not only for mechanical intervention but also as vehicles for providing biological therapy. Biological therapy uses medicated stents to locally administer an active agent or drug. Effective concentrations at the treated site require systemic drug administration which often produces adverse or even toxic side effects. Local delivery is a preferred treatment method because it administers smaller total medication levels than systemic methods, but concentrates the drug at a specific site. Local delivery thus produces fewer side effects and achieves better results.

A medicated stent may be fabricated by coating the surface of a stent with a drug or a drug and a polymeric carrier. Those of ordinary skill in the art fabricate coatings by applying a polymer, or a blend of polymers, to the stent using well-known techniques. Such a coating composition may include a polymer solution and a drug dispersed in the solution. The composition may be applied to the stent by immersing the stent in the composition or by spraying the composition onto the stent. The solvent then evaporates, leaving on the stent surfaces a polymer coating impregnated with the drug.

Coating integrity, such as adhesion of a coating on a stent, is an important parameter for medicated stents with drug coatings. Inadequate adhesion of a coating on a stent can result in tearing, delamination, peeling, and/or fracture. Such phenomena can lead to formation of emboli and poor uniformity of drug delivery to a vessel.

### SUMMARY

Certain embodiments of the present invention are directed to a method of coating a stent comprising: applying a coating material to a polymeric surface of a stent, the coating material including a coating polymer dissolved in a solvent, wherein the solvent is capable of swelling the surface polymer and is incapable or substantially incapable of dissolving the surface polymer; allowing the solvent to swell at least a portion of the surface polymer; and removing all or a substantial portion of the solvent from the applied coating material to form a coating on the stent.

Additional embodiments of the present invention are directed to a method of coating a stent comprising: applying a swelling solvent to a polymeric surface of a stent, wherein the swelling solvent is capable of swelling the surface polymer and is incapable or substantially incapable of dissolving the surface polymer; allowing the swelling solvent to swell at least a portion of the polymeric surface; applying a coating material to the swollen polymeric surface, the coating material including a coating polymer dissolved in a coating solvent; and removing all or a substantial portion of the swelling and the coating solvent from the surface polymer and the applied coating material to form a coating on the stent.

Further embodiments of the present invention are directed to a method of coating a stent comprising: forming a primer layer on a polymeric surface of a stent, wherein the primer layer is formed by applying a primer coating material to the polymeric surface of the stent, the primer coating material including a primer polymer dissolved in a primer solvent, wherein the primer solvent is capable of swelling the surface polymer and is incapable or substantially incapable of dissolving the surface polymer; by allowing the primer solvent to swell at least a portion of the surface polymer; and by removing all or a substantial portion of the primer solvent from the applied primer coating material to form the primer layer on the stent; and forming a drug layer over the primer layer, wherein the drug layer is formed by applying a drug coating material to a surface of the primer layer, the drug coating material comprising a drug dissolved in a drug solvent; and by removing all or a substantial portion of the drug solvent from the applied drug coating material.

Other embodiments of the present invention are directed to a method of coating a stent comprising: spraying a coating material for application onto a polymeric surface of a stent, the coating material including a coating polymer dissolved in a solvent, wherein the solvent is capable of swelling the surface polymer and is incapable or substantially incapable of dissolving the surface polymer; and modifying at least one process parameter of the spraying so that a weight percent of solvent in coating material applied onto the polymeric surface is less than about 15 wt %.

### BRIEF DESCRIPTION OF THE DRAWINGS

FIG. 1 depicts a stent.

FIG. 2A depicts a cross-section of a stent surface with a drug-polymer layer.

FIG. 2B depicts a cross-section of a stent surface with a primer layer and a drug-polymer layer.

FIG. 3A depicts a cross-section of a stent surface showing a coating material layer over a swollen surface polymer layer.

FIG. 3B depicts a cross-section of a stent surface showing a drug-polymer layer and an interfacial layer.

FIG. 4A depicts a cross-section of a stent surface showing a swollen surface polymer layer over an unswollen substrate or coating layer.

FIG. 4B depicts a cross-section of a stent surface showing a coating material layer over a swollen surface polymer layer.

FIG. 4C depicts a cross-section of a stent surface showing a coating layer over an interfacial layer that is above a substrate or a coating layer.

FIG. 5A depicts a cross-section of a stent surface showing a primer coating material layer over a swollen surface polymer.

FIG. 5B depicts a cross-section of a stent surface showing a primer coating layer and an interfacial layer.

FIG. 5C depicts a cross-section of a stent surface showing a drug layer above a primer layer that is above an interfacial layer.

FIG. 6 depicts an exemplary schematic embodiment of a spray coating apparatus for coating a stent.

### DETAILED DESCRIPTION OF THE INVENTION

Various embodiments of the present invention relate to improving adhesion of coatings applied on polymeric surfaces of stents. A polymeric surface may be a surface of a polymer coating disposed over a substrate composed of metal, polymer, ceramic, or other suitable material. Alternatively, a surface may be a surface of a polymeric substrate of a stent.

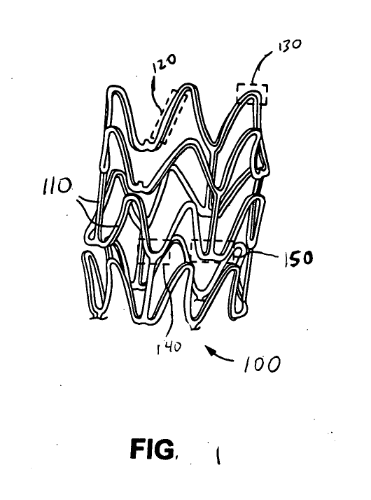

The present invention may be applied to implantable medical devices including, but not limited to, self-expandable stents, balloon-expandable stents, stent-grafts, and grafts (e.g., aortic grafts). A stent can have a scaffolding or a substrate that includes a pattern of a plurality of interconnecting structural elements or struts. FIG. 1 depicts an example of a view of a stent 100. Stent 100 includes a pattern with a number of interconnecting structural elements or struts 110. In general, a stent pattern is designed so that the stent can be radially compressed (crimped) and radially expanded (to allow deployment). The stresses involved during compression and expansion are generally distributed throughout various structural elements of the stent pattern.

As shown in FIG. 1, the geometry or shape of stent 100 varies throughout its structure to allow radial expansion and compression. A pattern may include portions of struts that are straight or relatively straight, an example being a portion 120. In addition, patterns may include bending elements 130, 140, and 150. Bending elements bend inward when a stent is crimped to allow radial compression. Bending elements also bend outward when a stent is expanded to allow for radial expansion. The present invention is not limited to the stent pattern depicted in FIG. 1. The variations in stent patterns is virtually unlimited.

In some embodiments, a stent may be fabricated by laser cutting a pattern on a tube or a sheet rolled into a tube. Representative examples of lasers that may be used include, but are not limited to, excimer, carbon dioxide, and YAG. In other embodiments, chemical etching may be used to form a pattern on a tube.

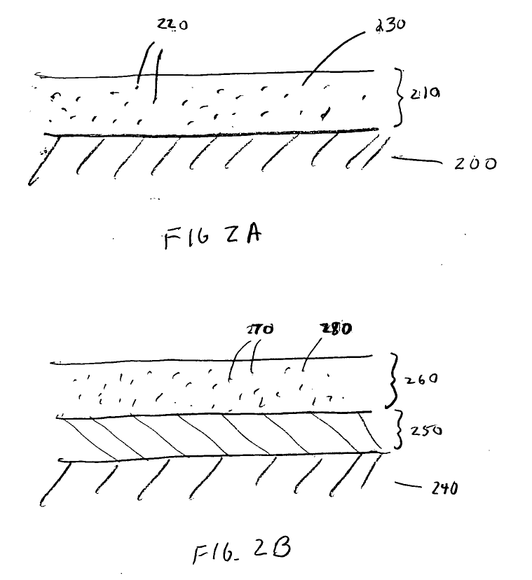

As indicated above, a medicated stent may be fabricated by coating the surface of a stent with a drug. For example, a stent can have a coating including a drug dispersed in a polymeric carrier disposed over a substrate. FIG. 2A depicts a cross-section of a stent surface with a drug-polymer coating layer 210 over a substrate 200. In other embodiments, drug-polymer layer 210 can be disposed over a polymeric coating layer. In some embodiments, coating layer 210 can also be pure drug. Coating layer 210 includes a drug 220 dispersed in a coating polymer 230. As indicated above, a substrate or scaffolding can be metallic, polymeric, ceramic, or other suitable material.

FIG. 2A depicts a cross-section of a substrate 240 of a stent with a polymeric layer 250 disposed over substrate 240. A drug-polymer coating layer 260 is disposed over polymeric layer 250. Coating layer 260 includes a drug 270 dispersed within a polymer 280. Polymeric layer 250 can be a primer layer for improving the adhesion of drug-polymer layer 260 to substrate 240.

As indicated above, a coating layer may be formed by applying a coating material to a stent. The coating material can be a polymer solution and a drug dispersed in the solution. The coating material may be applied to the stent by immersing the stent in the coating material, by spraying the composition onto the stent, or by other methods known in the art. The solvent in the solution then evaporates, leaving on the stent surfaces a polymer coating impregnated with the drug. In other embodiments, the coating material can include a drug dispersed or dissolved in a solvent without a polymer.

Stents are typically subjected to stress during use, both before and during treatment. “Use” includes manufacturing, assembling (e.g., crimping a stent on balloon), delivery of a stent through a bodily lumen to a treatment site, and deployment of a stent at a treatment site. Both the underlying scaffolding or substrate and the coating experience stress that result in strain in the substrate and coating. In particular, localized portions of the stent's structure undergo substantial deformation. For example, the apex regions of bending elements 130, 140, and 150 in FIG. 1 experience relatively high stress and strain during crimping, expansion, and after expansion of the stent.

Furthermore, polymer substrates or polymer-based coatings may be particularly vulnerable to mechanical instability during use of a stent. Polymers, in general, and many polymers used in coatings for devices tend to have a relatively high degree of inelasticity, and, hence have relatively low strength compared to a metal. Therefore, polymer-based coatings are highly susceptible to tearing or fracture, and/or detachment, especially at regions subjected to relatively high stress and strain.

In certain embodiments, the method of enhancing coating integrity or adhesion of a coating to a polymeric surface of a stent can include applying a coating material to the polymeric surface of a stent in which the coating material includes a coating polymer dissolved in a solvent. The coating material can also include a drug mixed or dispersed in the coating material. In an embodiment, the surface polymer is capable of being swollen by the solvent and has a relatively low or no solubility in the solvent.

As is understood by persons of skill in the art, swelling of a polymer occurs when a solvent in contact with a sample of the polymer diffuses into the polymer. L. H. Sperling, Physical Polymer Science, 3^{rd} ed., Wiley (2001). Thus, a swollen polymer sample includes solvent molecules dispersed within the bulk of the polymer. Dissolution of the polymer occurs when polymer molecules diffuse out of the swollen polymer into solution.

The phrase “the solvent is capable of swelling the surface polymer and is incapable or substantially incapable of dissolving the surface polymer” is understood to mean a sample of the surface polymer swells when immersed in the solvent and the swollen sample of the surface polymer remains in the solvent with a negligible loss of mass for an indefinite period of time at conditions of ambient temperature and temperature. Specifically, a “solvent” for a given polymer can be defined as a substance capable of dissolving or dispersing the polymer or capable of at least partially dissolving or dispersing the polymer to form a uniformly dispersed mixture at the molecular- or ionic-size level. The solvent should be capable of dissolving at least 0.1 mg of the polymer in 1 ml of the solvent, and more narrowly 0.5 mg in 1 ml at ambient temperature and ambient pressure.

A substance incapable or substantially incapable of dissolving a polymer should be capable of dissolving only less than 0.1 mg of the polymer in 1 ml of the non-solvent at ambient temperature and ambient pressure, and more narrowly only less than 0.05 mg in 1 ml at ambient temperature and ambient pressure. A substance incapable or substantially incapable of dissolving a given polymer is generally referred to as a nonsolvent for that polymer.

Solvents and nonsolvents for polymers can be found in standard texts (e.g., see Fuchs, in Polymer Handbook, 3rd Edition and Deasy, Microencapsulation and Related Drug Processes, 1984, Marcel Dekker, Inc., New York.) The ability of a polymer to swell and to dissolve in a solvent can be estimated using the Cohesive Energy Density Concept (CED) and related solubility parameter values as discussed by Deasy and can be found in detail in the article by Grulke in Polymer Handbook. Thus, a person skilled in the art will be able to select a solvent that “is capable of swelling the surface polymer and is incapable or substantially incapable of dissolving the surface polymer.”

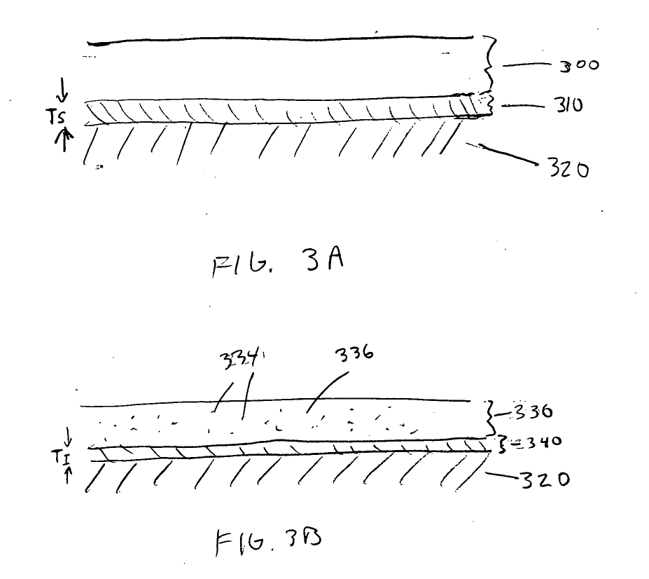

Additionally, the method may include allowing the solvent to swell at least a portion of the surface polymer. In an embodiment, the applied solvent may form swollen layer of surface polymer over unswollen surface polymer. FIG. 3A depicts a cross-section of a stent showing a coating material layer 300 over a swollen surface polymer layer 310. Swollen surface polymer layer 310 is over unswollen polymer coating layer or polymer substrate 320. As indicated above, unswollen surface polymer 320 can either be a substrate of the stent or a polymeric coating over a stent substrate. As shown, swollen surface polymer layer 310 has a thickness Ts.

A coating on the stent may then be formed by removing all or a substantial portion of the solvent from the applied coating material. In particular, all or a substantial portion of the solvent is removed from coating material layer 300 and swollen layer 310.

Drying or solvent removal can be performed by allowing the solvent to evaporate at room or ambient temperature. Depending on the volatility of the particular solvent employed, the solvent can evaporate essentially upon contact with the stent. Alternatively, the solvent can be removed by subjecting the coated stent to various drying processes. Drying time can be decreased to increase manufacturing throughput by heating the coated stent. For example, removal of the solvent can be induced by baking the stent in an oven at a mild temperature (e.g., 60° C.) for a suitable duration of time (e.g., 2-4 hours) or by the application of warm air. In an embodiment, a substantial portion of solvent removed may correspond to less than 5%, 3%, or more narrowly, less than 1% of solvent remaining after drying.

Depositing a coating of a desired thickness in a single coating stage can result in an undesirably nonuniform surface structure and/or coating defects. Therefore, a coating process can involve multiple repetitions of application, for example, by spraying, forming a plurality of layers. Thus, swelling of the surface polymer may tend to occur in application of the first coating layer. However, in some embodiments, swelling may occur upon application of coating layers after the first layer. The occurrence of such swelling depends in part upon the thickness of the layers and the amount of solvent remaining in coating layers after drying.

Due to swelling of the surface polymer in swollen polymer layer 310, it is believed that the polymer chains of the coating polymer in coating layer 300 penetrate into or mix with the surface polymer in swollen polymer layer 310 prior to removal of the solvent. As depicted in FIG. 3B, upon removal of the solvent, a coating layer 330 is formed that includes drug 334 dispersed within coating polymer 336. In addition, it is believed that there is an interfacial layer 340 that includes coating polymer 336 and surface polymer. Thus, there may be a gradual transition in composition between coating layer 330 and the substrate or coating layer 320, which is composed of surface polymer. It is expected that interfacial layer 340 can improve or enhance adhesion of coating layer 330 onto substrate or coating layer 320. As shown, interfacial layer 340 has a thickness Ti.

Additionally, the enhanced adhesion due to the interfacial layer may allow greater flexibility in the concentration of drug in a drug layer. For many drug-polymer systems, the presence of drug in a drug-polymer coating can reduce the flexibility of the polymer. The polymer can even become brittle at high enough drug concentration. The reduced flexibility or brittleness of the polymer can make the drug-polymer coating more susceptible to tearing, delamination, peeling, and/or fracture. The enhanced adhesion may reduce or prevent such coating failure which can allow higher drug concentration in a drug-polymer coating.

An exemplary embodiment corresponding to FIGS. 3A and 3B includes a stent with a polymeric substrate composed of poly(L-lactide) (PLLA). The PLLA substrate can be coated with a coating material including poly(DL-lactide) (PDLA) dissolved in acetone. Acetone swells, but does not dissolve PLLA.

Additionally, it is likely that the greater the thickness Ti, the greater the enhancement of the adhesion of applied coating layer 330 to substrate or coating layer 320. However, the swelling of surface polymer of substrate or coating layer 320 can have deleterious effects, which can make limiting the size of thickness Ti desirable. In particular, substrate 320 may have selected mechanical properties that allow it to serve as a structural support for the stent. Swelling of the surface polymer with subsequent removal solvent can adversely effect the mechanical properties of the surface polymer in the interfacial layer 340. As a result, the mechanical properties of interfacial layer 340 can be less desirable for use as structural support. For example, a polymeric stent substrate may have a high radial strength due to alignment of polymer chains along a circumferential direction. Swelling of the substrate may reduce or eliminate the alignment, resulting in a loss of radial strength.

Thickness Ti of interfacial layer 340 is directly related to thickness Ts of swollen layer 310. Thickness Ts of swollen layer 310 depends at least in part on the fraction of the solvent in applied coating material. It is expected that the higher the fraction of solvent in the applied coating material, the greater the thickness Ts of swollen layer 310, and the greater the resulting thickness Ti of interfacial layer 340. Thus, thickness Ts and thickness Ti can be controlled by controlling the fraction of the solvent in applied coating material. An acceptable degree of adhesion can be obtained by having a weight percent of solvent in the applied coating material that is sufficient to swell at least a surface layer of the substrate polymer. The weight percent of solvent in applied coating material may be controlled by modifying the parameters of a coating material application method.

For example, parameters in an immersion coating process include the temperature of the coating material solution. Increasing the temperature of the coating material solution increases the weight percent of polymer in solution, thus decreasing the weight percent of solvent. Modifying parameters of a spray coating process are described below.

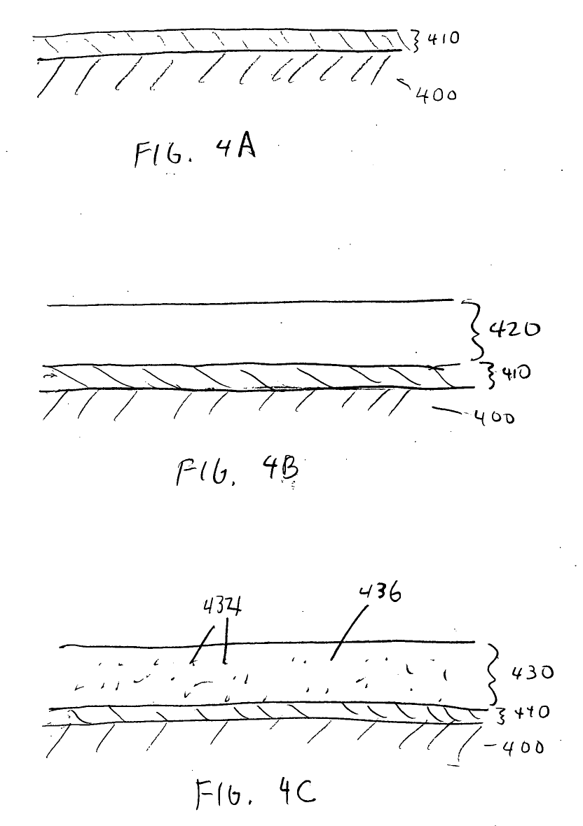

In some embodiments, it may be advantageous to swell (or pre-swell) a polymeric substrate or polymeric coating layer prior to applying a coating material that includes a coating polymer and/or a drug. Embodiments of a method involving pre-swelling can include applying a swelling solvent to a polymeric surface of a stent such that the swelling solvent is capable of swelling the surface polymer and is incapable or substantially incapable of dissolving the surface polymer. The method may further include allowing the swelling solvent to swell at least a portion of the polymeric surface. For example, FIG. 4A depicts a swollen layer 410 over an unswollen substrate or coating layer 400. Swollen layer layer 410 includes surface polymer swollen by the swelling solvent while substrate or coating layer 400 includes unswollen surface polymer.

Additionally, the method can include applying a coating material to the swollen polymeric surface such that the coating material includes a coating polymer dissolved in a coating solvent and optionally a drug mixed or dispersed in the coating material. FIG. 4B shows coating material layer 420 disposed over swollen layer 410. All or substantially all of the swelling and the coating solvent can then be removed from the surface polymer and applied coating material to form a coating on the stent. FIG. 4C depicts a coating layer 430 over an interfacial layer 440, having properties as described above, and a substrate or coating layer 400. Coating layer 430 has a drug 434 mixed or dispersed in a coating polymer 436. In general, it is desirable for the swelling solvent and the coating solvent to be substantially or completely immiscible.

An exemplary embodiment corresponding to FIGS. 4A and 4B includes a stent with a polymeric substrate composed of poly(L-lactide) (PLLA). The PLLA substrate can be pre-swollen with chloroform. The swollen PLLA substrate can then be coated with a coating material including poly(DL-lactide) (PDLA) dissolved in ethanol. In another exemplary embodiment, PLLA substrate can be pre-swollen with acetone. The swollen PLLA substrate can then be coated with a coating material including polyethylene glycol dissolved in water.

Pre-swelling can be particularly advantageous since the coating material solvent and the swelling solvent need not be the same solvent. The use of a different solvent for the coating material and the swelling can provide a degree of flexibility to the coating process, as described below.

Generally, a treatment with a medicated stent may require a particular drug coating on a coating of a medicated stent. A drug may have an undesirably low or negligible solubility in a selected group of solvents that can swell the surface polymer. Thus, a drug coating formed using such swelling solvent can have an undesirably low concentration of drug. Thus, a suitable solvent can be used to swell the surface polymer and different solvent can be used as a coating solvent, in which the drug has an acceptable solubility. In general, a required solubility of a drug in a coating solvent is determined by the drug loading required of a particular treatment regimen. Specifically, it is desirable for a drug to have solubility of at least 1 wt % in a solvent for use as a coating material solvent for forming a drug-polymer layer on a stent.

In addition, there is also flexibility relating to the miscibility of the swelling solvent and the coating solvents. The solvents can be selected to have a desired degree of miscibility. For example, the solvents can be selected so that they have a relatively low miscibility or are immiscible. The use of immiscible coating and swelling solvents may allow greater control of the degree of swelling of substrate or coating layer 400. If the solvents are immiscible, the swelling solvent will not mix with the applied coating material.

However, if the solvents are miscible, swelling solvent will mix with coating solvent, reducing the concentration of the swelling solvent in contact with the surface polymer. As a result, the degree of swelling of the surface polymer will be reduced if the coating solvent is a weaker solvent for the surface polymer.

Other embodiments of a method of enhancing adhesion can include forming a primer layer over a polymer substrate or coating layer, and then forming a drug-polymer coating layer over the primer layer. In certain embodiments, the primer layer may be formed by applying a primer coating material to a polymeric surface of the stent. The primer coating material can include a primer polymer dissolved in a primer solvent such that the primer solvent is capable of swelling the surface polymer and is incapable or substantially incapable of dissolving the surface polymer.

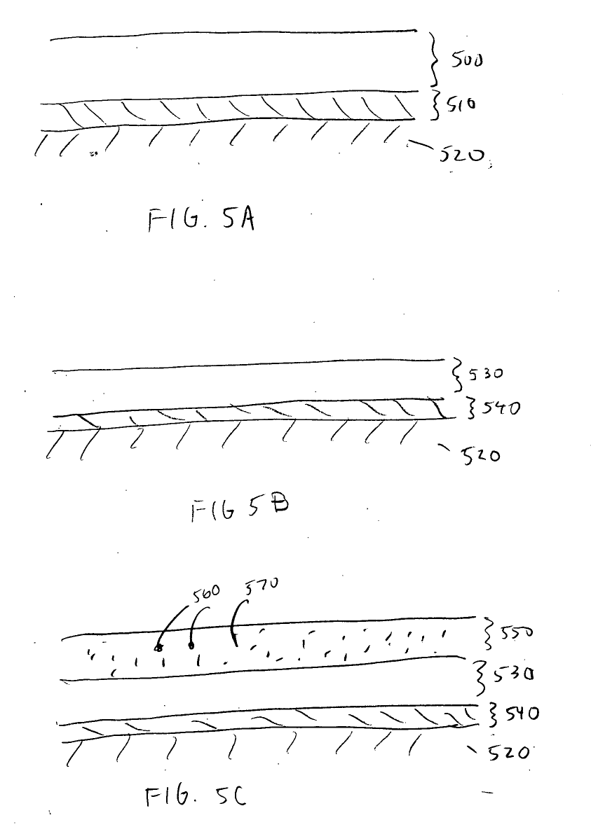

Forming the primer layer further includes allowing the primer solvent to swell at least a portion of the surface polymer. FIG. 5A depicts a cross-section of a surface of a stent showing a primer coating material layer 500 over a swollen surface polymer layer 510. Swollen surface polymer layer 510 is over unswollen polymer coating layer or polymer substrate 520.

All or substantially all of the primer solvent may then be removed from the applied primer coating material to form the primer layer on the stent. FIG. 5B shows, upon removal of the solvent, a primer coating layer 530 is formed that includes the primer polymer. An interfacial layer 540, discussed above, includes primer polymer and surface polymer.

Additionally, a drug layer may then be formed over the primer layer by applying a drug coating material to a surface of the primer layer. The drug coating material may include a drug dissolved in a drug solvent. Also, the drug coating material may also include a polymer, different from the primer polymer, dissolved in the drug solvent. All or substantially all of the drug solvent may be removed from the applied drug coating material to form the drug layer. FIG. 5C depicts a drug layer 550 over primer coating layer 530. Drug layer 550 includes a drug 560 mixed or dispersed within a polymer 570.

The embodiments depicted in FIGS. 5A-C may be advantageous when a drug has an undesirably low or negligible solubility in a selected group of solvents that can swell, but not dissolve the surface polymer. Such solvents, as discussed above, can be unsuitable for use in forming a drug layer. Thus, one of the swelling solvents can be used to enhance adhesion of a primer layer to a coating layer or substrate and another more suitable solvent can be used to form the drug layer over the primer layer. As depicted in FIGS. 5A-C, an interfacial region 540 enhances the adhesion of primer layer 530 to substrate or coating layer 520 and indirectly enhances adhesion of drug layer 550 to substrate or coating layer 520.

An exemplary embodiment corresponding to FIGS. 5A-C includes a stent with a polymeric substrate composed of polyglycolide (PGA). A primer layer composed of 50/50 poly(DL-lactide-co-glycolide) (PDLA-co-GA) is disposed over the PGA. A drug layer of everolimus is disposed over the primer layer. The primer layer can be formed by applying a solution of PDLA-co-GA dissolved in hexafluoroisopropanol (HFIP). HFIP can swell, but does not dissolve PGA. However, HFIP is a poor solvent for everolimus. The drug layer can be formed by applying a solution of everolimus in acetone.

Further embodiments of the present invention can include controlling the fraction of swelling solvent in a coating material applied to a polymeric surface of a stent. In some embodiments, the coating material can be applied by spraying the coating material onto the polymeric surface of the stent. The coating material may include a coating polymer dissolved in a swelling solvent. As describe above, the swelling solvent is capable of swelling the surface polymer and not dissolving the surface polymer.

As discussed above, it may be desirable to control the amount of surface polymer that is swelled. In general, increasing the fraction of swelling solvent in the coating material increases the amount of surface polymer swelled, which results in a greater swelling layer thickness Ts, as shown in FIG. 3A. In some embodiments, the method of coating may include modifying at least one process parameter of the spraying so that a weight percent of solvent in coating material applied on the polymeric surface is less than about 30 wt %, 20 wt %, 15 wt %, or more narrowly, 10 wt %.

Spray coating a stent typically involves mounting or disposing a stent on a support, followed by spraying a coating material from a nozzle onto the mounted stent. A spray apparatus, such as EFD 780S spray device with VALVEMATE 7040 control system (manufactured by EFD Inc., East Providence, R. I., can be used to apply a coating material to a stent. An EFD 780S spray device is an air-assisted external mixing atomizer. The coating material is atomized into small droplets by air and uniformly applied to the stent surfaces. Other types of spray applicators, including air-assisted internal mixing atomizers and ultrasonic applicators, can also be used for the application of the coating material. To facilitate uniform and complete coverage of the stent during the application of the composition, the stent can be rotated about the stent's central longitudinal axis. The stent can also be moved in a linear direction along the same axis.

A nozzle can deposit coating material onto a stent in the form of fine droplets. The droplet size depends on factors such as viscosity of the solution, surface tension of the solvent, and atomization pressure. Only a small percentage of the composition that is delivered from the spray nozzle is ultimately deposited on the stent.

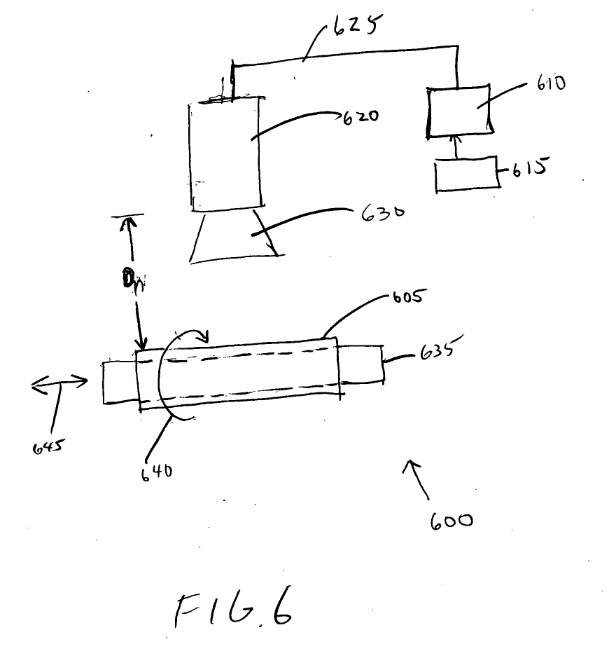

FIG. 6 depicts an exemplary schematic embodiment of a spray coating apparatus 600 for coating a stent 605. A syringe pump 610 pumps coating material from a reservoir 615 that is in fluid communication with a spray nozzle 620. Nozzle 610 can be in fluid communication with pump 610 through a hose 625. Nozzle 620 provides a plume 630 of fine droplets of coating material for depositing on stent 605. Nozzle 620 is positioned a distance Dn form the surface of stent 605. A flow rate of coating material provided by nozzle 610 can be varied by changing the pump rate of pump 610.

Stent 605 is supported by a stent support 635, such as a mandrel. Support 635 can be configured to rotate stent 605 about its cylindrical axis, as shown by an arrow 640. Support 635 can also be configured to axially or linearly translate stent 605 with respect to plume 630, as shown by an arrow 645.

A number of spray process parameters can influence the fraction of solvent in the coating material that is applied or deposited on stent 605. These process parameters include, but are not limited to, the atomization temperature, the atomization pressure, the temperature of the atomized coating material between the nozzle and the stent, and the pressure of the atomized coating material between the nozzle and the stent.

With respect to temperature, increasing the atomization temperature and temperature of the atomized coating material between the nozzle and the stent tends to decrease the fraction of solvent in the coating material. Increasing the temperature will cause evaporation of solvent from the coating material resulting in a decrease in the fraction of solvent in the coating material. A nozzle can be equipped with a heating element to heat the coating material before and/or during atomization above an ambient temperature. In addition, the atomized coating material and the coating material applied to the stent can be heated. For example, heat nozzles can blow a heated gas on the coating material between the nozzle and the stent and on the stent. Both the temperature and pressure of heated gas can also affect the evaporation of solvent from the coating material.

Additionally, decreasing the atomization pressure can also decrease the fraction of solvent in the coating material. Also, the spray coating apparatus can be enclosed in a chamber to allow control of the pressure of atomized coating material. Reducing the chamber pressure, for example, to below ambient pressure will reduce the fraction of solvent in the atomized coating material.

Additional parameters that can be used to control the fraction of solvent in applied coating material include the flow rate of the coating material, distance Dn, and the size of droplets of atomized droplets. Increasing distance Dn decreases the fraction of solvent in coating material applied to the stent since the time for evaporation of solvent from the falling droplets is increased. In addition, there is a higher evaporation rate of smaller atomized droplets due to a higher surface to volume ratio. As a result, smaller droplet size results in a lower fraction of solvent in the applied coating material. The droplet size can be controlled, for example, by nozzle design. One of skill in the art could select a nozzle that could result in smaller droplets. Additionally, reducing the flow rate of coating material tends to result in smaller atomized coating material droplets which tends to increase the evaporation rate of solvent.

In an exemplary embodiment, a stent having a substrate of PLLA is coated with PDLA. The coating material is PDLA dissolved in acetone. The weight fraction of solvent in coating. material can be greater than 50%, 70%, 80%, 95%, or more narrowly, 97%. The spray nozzle temperature or atomization temperature can be between about 15° C. and 30° C. Atomization pressure can be between 5.5 psi and 7 psi. A temperature of heated air from a heat nozzle directed at the stent can be between 38° C. and 40° C. The air pressure of the nozzle can be between 18 psi and 22 psi. The syringe pump rate can be between 2 ml/hr and 6 ml/hr.

A drug or active agent can include, but is not limited to, any substance capable of exerting a therapeutic, prophylactic, or diagnostic effect. The drugs for use in the implantable medical device, such as a stent or non-load bearing scaffolding structure may be of any or a combination of a therapeutic, prophylactic, or diagnostic agent. Examples of active agents include antiproliferative substances such as actinomycin D, or derivatives and analogs thereof (manufactured by Sigma-Aldrich 1001 West Saint Paul Avenue, Milwaukee, Wis. 53233; or COSMEGEN available from Merck). Synonyms of actinomycin D include dactinomycin, actinomycin IV, actinomycin I₁, actinomycin X₁, and actinomycin C₁. The bioactive agent can also fall under the genus of antineoplastic, anti-inflammatory, antiplatelet, anticoagulant, antifibrin, antithrombin, antimitotic, antibiotic, antiallergic and antioxidant substances. Examples of such antineoplastics and/or antimitotics include paclitaxel, (e.g., TAXOL® by Bristol-Myers Squibb Co., Stamford, Conn.), docetaxel (e.g., Taxotere®, from Aventis S. A., Frankfurt, Germany), methotrexate, azathioprine, vincristine, vinblastine, fluorouracil, doxorubicin hydrochloride (e.g., Adriamycin® from Pharmacia & Upjohn, Peapack N.J.), and mitomycin (e.g., Mutamycin® from Bristol-Myers Squibb Co., Stamford, Conn.). Examples of such antiplatelets, anticoagulants, antifibrin, and antithrombins include aspirin, sodium heparin, low molecular weight heparins, heparinoids, hirudin, argatroban, forskolin, vapiprost, prostacycl in and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax ä (Biogen, Inc., Cambridge, Mass.). Examples of such cytostatic or antiproliferative agents include angiopeptin, angiotensin converting enzyme inhibitors such as captopril (e.g., Capoten® and Capozide® from Bristol-Myers Squibb Co., Stamford, Conn.), cilazapril or lisinopril (e.g., Prinivil® and Prinzide® from Merck & Co., Inc., Whitehouse Station, N.J.), calcium channel blockers (such as nifedipine), colchicine, proteins, peptides, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents which may be appropriate agents include cisplatin, insulin sensitizers, receptor tyrosine kinase inhibitors, carboplatin, alpha-interferon, genetically engineered epithelial cells, steroidal anti-inflammatory agents, non-steroidal anti-inflammatory agents, antivirals, anticancer drugs, anticoagulant agents, free radical scavengers, estradiol, antibiotics, nitric oxide donors, super oxide dismutases, super oxide dismutases mimics, 4-amino-2,2,6,6-tetramethylpiperidine-1-oxyl (4-amino-TEMPO), tacrolimus, dexamethasone, ABT-578, clobetasol, cytostatic agents, prodrugs thereof, co-drugs thereof, and a combination thereof. Other therapeutic substances or agents may include rapamycin and structural derivatives or functional analogs thereof, such as 40-O-(2-hydroxy)ethyl-rapamycin (known by the trade name of EVEROLIMUS), 40-O-(3-hydroxy)propyl-rapamycin, 40-O-[2-(2-hydroxy)ethoxy]ethyl-rapamycin, methyl rapamycin, and 40-O-tetrazole-rapamycin.

Representative examples of solvents that may be used in accordance with the present invention include, but are not limited to, acetone, chloroform, hexafluoroisopropanol, 1,4-dioxane, tetrahydrofuran (THF), dichloromethane acetonitrile, dimethyl sulfoxide (DMSO), and dimethylformamide (DMF), cyclohexane, toluene, xylene, acetone, ethyl acetate.

A stent substrate can be fabricated from a biostable metal, a bioerodible metal, or combination thereof. Representative bioerodible metals include, but are not limited to, magnesium, zinc, and iron. Representative biostable metals include, but are not limited to, metallic materials or an alloys such as cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum.

A polymer for use in fabricating a substrate of a stent or a coating for a stent subtrate can be biostable, bioabsorbable, biodegtadable or bioerodable. Biostable refers to polymers that are not biodegradable. The terms biodegradable, bioabsorbable, and bioerodable are used interchangeably and refer to polymers that are capable of being completely degraded and/or eroded when exposed to bodily fluids such as blood and can be gradually resorbed, absorbed and/or eliminated by the body. The processes of breaking down and absorption of the polymer can be caused by, for example, hydrolysis and metabolic processes.

It is understood that after the process of degradation, erosion, absorption, and/or resorption has been completed, no part of the stent will remain or in the case of coating applications on a biostable scaffolding, no polymer will remain on the device. In some embodiments, very negligible traces or residue may be left behind. For stents made from a biodegradable polymer, the stent is intended to remain in the body for a duration of time until its intended function of, for example, maintaining vascular patency and/or drug delivery is accomplished.

Representative examples of polymers that may be used to fabricate a substrate or a coating for a stent substrate include, but are not limited to, poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(L-lactide-co-glycolide); poly(D,L-lactide), poly(caprolactone), poly(trimethylene carbonate), polyethylene amide, polyethylene acrylate, poly(glycolic acid-co-trimethylene carbonate), co-poly(ether-esters) (e.g. PEOIPLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

Additional representative examples of polymers that may be especially well suited for use in fabricating an implantable medical device according to the methods disclosed herein include ethylene vinyl alcohol copolymer (commonly known by the generic name EVOH or by the trade name EVAL), poly(butyl methacrylate), poly(vinylidene fluoride-co-hexafluororpropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, and polyethylene glycol.

While particular embodiments of the present invention have been shown and described, it will be obvious to those skilled in the art that changes and modifications can be made without departing from this invention in its broader aspects.

What is claimed is:

1. A method of coating a stent comprising:
applying a coating material to a polymeric surface of a stent, the coating material including a coating polymer dissolved in a solvent, wherein the solvent is capable of swelling the surface polymer and incapable or substantially incapable of dissolving the surface polymer;
allowing the solvent to swell at least a portion of the surface polymer; and
removing all or a substantial portion of the solvent from the applied coating material to form a coating on the stent.

2. The method of claim 1, wherein the coating material further comprises a therapeutic agent.

3. The method of claim 1, wherein the surface comprises a surface of a coating layer including the surface polymer disposed over a substrate of the stent.

4. The method of claim 1, wherein the surface comprises a surface of a substrate of the stent, the substrate comprising the surface polymer.

5. The method of claim 1, further comprising controlling parameters of the application of coating material so that the weight percent of solvent in the coating material applied onto the polymeric surface is less than about 15 wt %.

6. The method of claim 1, wherein the weight percent of solvent in the coating material applied onto the polymeric surface is less than about 15 wt %.

7. The method of claim 1, wherein the surface polymer is a biostable polymer, biodegradable polymer, or a combination thereof.

8. The method of claim 1, wherein the coating polymer is a biostable polymer, biodegradable polymer, or a combination thereof.

9. A method of coating a stent comprising:
applying a swelling solvent to a polymeric surface of a stent, wherein the swelling solvent is capable of swelling the surface polymer and is incapable or substantially incapable of dissolving the surface polymer;
allowing the swelling solvent to swell at least a portion of the polymeric surface;
applying a coating material to the swollen polymeric surface, the coating material including a coating polymer dissolved in a coating solvent; and
removing all or a substantial portion of the swelling and the coating solvent from the swollen surface polymer and the applied coating material to form a coating on the stent.

10. The method of claim 9, wherein the coating solvent is not capable of dissolving or swelling the surface polymer.

11. The method of claim 9, wherein the coating material further comprises a drug

12. The method of claim 11, wherein the drug is insoluble in the swelling solvent.

13. The method of claim 9, wherein the swelling solvent and the coating solvent are immiscible.

14. The method of claim 9, wherein the surface polymer is a biostable polymer, biodegradable polymer, or a combination thereof.

15. The method of claim 9, wherein the coating polymer is a biostable polymer, biodegradable polymer, or a combination thereof.

16. A method of coating a stent comprising:
forming a primer layer on a polymeric surface of a stent, wherein the primer layer is formed by applying a primer coating material to the polymeric surface of the stent, the primer coating material including a primer polymer dissolved in a primer solvent, wherein the primer solvent is capable of swelling the surface polymer and is incapable or substantially incapable of dissolving the surface polymer; by allowing the primer solvent to swell at least a portion of the surface polymer; and by removing all or a substantial portion of the primer solvent from the applied primer coating material to form the primer layer on the stent; and
forming a drug layer over the primer layer, wherein the drug layer is formed by applying a drug coating material to a surface of the primer layer, the drug coating material comprising a drug dissolved in a drug solvent; and by removing all or a substantial portion of the drug solvent from the applied drug coating material.

17. The method of claim 16, wherein the drug is insoluble is the primer solvent.

18. The method of claim 16, wherein the drug coating material further comprises a third polymer so that the drug layer comprises the drug and a third polymer.

19. The method of claim 16, wherein the surface comprises a surface of a coating layer including the surface polymer disposed over a substrate of the stent.

20. The method of claim 16, wherein the surface comprises a surface of a substrate of the stent, the substrate comprising the surface polymer.

21. The method of claim 16, wherein the surface polymer is a biostable polymer, biodegradable polymer, or a combination thereof.

22. The method of claim 16, wherein the primer polymer is a biostable polymer, biodegradable polymer, or a combination thereof.

23. A method of coating a stent comprising:
spraying a coating material for application onto a polymeric surface of a stent, the coating material including a coating polymer dissolved in a solvent, wherein the solvent is capable of swelling the surface polymer and is incapable or substantially incapable of dissolving the surface polymer; and
modifying at least one process parameter of the spraying so that a weight percent of solvent in coating material applied onto the polymeric surface is less than about 15 wt %.

24. The method of claim 23, further comprising allowing the solvent to swell at least a portion of the surface polymer; and removing all or a substantial portion of the solvent from the applied coating material to form a coating on the stent.

25. The method of claim 23, wherein at least one process parameter is selected from the group consisting of a temperature of the coating material during spraying and deposition, pressure, flow rate of the sprayed coating material, distance between a nozzle from which coating material is sprayed and the polymeric surface, and size of droplets of sprayed coating material.
